(19) United States
(12) Patent Application Publication
(10) Pub. No.: US 2009/0191245 A1
(43) Pub. Date: Jul. 30, 2009

(54) REDUCED-IRRITANT DERMATOLOGICAL COMPOSITIONS COMPRISING AT LEAST ONE NAPHTHOIC ACID COMPOUND AND BENZOYL PEROXIDE AND TREATMENT OF KERATINIZATION DISORDERS THEREWITH

(75) Inventors: Laurent Fredon, Roquefort Les Pins (FR); Nathalie Willcox, Saint Vallier De Thiey (FR); Sandrine Segura-Orsoni, Mandelieu (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/272,061

(22) Filed: Nov. 17, 2008

Related U.S. Application Data
(63) Continuation of application No. PCT/FR2007/051296, filed on May 16, 2007.

(30) Foreign Application Priority Data
May 17, 2006 (FR) 0604407

(51) Int. Cl. A61K 8/36; A61K 9/14; A61K 31/192

(52) U.S. Cl. 424/401; 424/490; 514/569; 424/497

(57) ABSTRACT

Topically applicable, reduced-irritant dermatological/cosmetic compositions useful for the prevention/treatment of a variety of keratinization disorders, for example acne vulgaris, contain at least one naphthoic acid compound and an amount of benzoyl peroxide encapsulated in a polymeric system of porous particles for increasing cutaneous penetration of the at least one naphthoic acid compound, are formulated into topically applicable, physiologically acceptable media therefor.

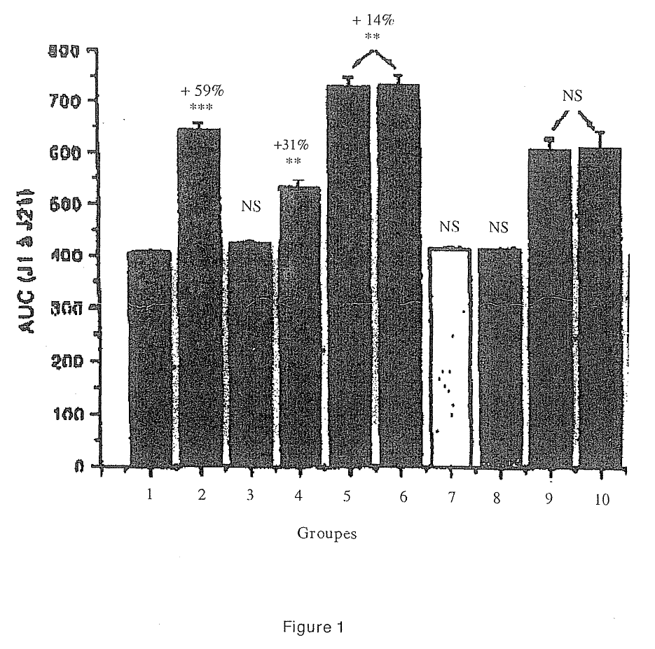

### CROSS-REFERENCE TO PRIORITY/PCT APPLICATIONS

This application claims priority under 35 U.S.C. § 119 of FR 06/04407, filed May 17, 2006, and is a continuation/national phase of PCT/FR 2007/051296, filed May 16, 2007 and designating the United States (published in the French language on Nov. 22, 2007 as WO 2007/132134 A2; the title and abstract were also published in English), each hereby expressly incorporated by reference in its entirety and each assigned to the assignee hereof.

### BACKGROUND OF THE INVENTION

1. Technical Field of the Invention

The present invention relates to dermatological compositions for topical application and to their applications as cosmetic or pharmaceutical products, such compositions being particularly useful for the treatment of acne.

2. Description of Background and/or Related and/or Prior Art

Acne is a common multifactor pathology which affects skin rich in sebaceous glands (face, scapular region, arms and intertriginous regions). It is the commonest form of dermatosis. The following five pathogenic factors play a determining role in the formation of acne:

1. genetic predisposition;

2. overproduction of sebum (seborrhoea);

3. androgens;

4, follicular keratinization disorders (comedogenesis); and

5. bacterial colonization and inflammatory factors.

Several forms of acne exist, all having it in common that the pilosebaceous follicles are attacked. Especially exemplary are acne conglobata, acne keloid on the back of the neck, acne medicamentosa, recurrent acne miliaria, acne necrotica, acne neonatorum, premenstrual acne, occupational acne, acne rosacea, senile acne, solar acne and acne vulgaris.

Acne vulgaris, also known as polymorphous juvenile acne, is the commonest. It comprises four stages but it is not necessary to pass through all the stages:

Stage 1 corresponds to comedonal acne, characterized by a large number of open and/or closed comedones and of microcysts.

Stage 2, or papulopustular acne, is of mild to moderate seriousness. It is characterized by the presence of open and/or closed comedones and of microcysts but also of red papules and of pustules. It mainly affects the face and leaves few scars.

Stage 3, or papulocomedonal acne, is more serious and extends to the back, to the thorax and to the shoulders. It is accompanied by a larger number of scars.

Stage 4, or nodulocystic acne, is accompanied by numerous scars. It exhibits nodules and also large painful purplish pustules.

The various forms of acne described above can be treated with active principles, such as anti-seborrhoeics and anti-infectives, for example, benzoyl peroxide (in particular, the product Eclaran® marketed by Pierre Fabre), with retinoids, such as tretinoin (in particular, the product Retacnyl® marketed by Galderma) or isotretinoin (product Roaccutane® marketed by Laboratoires Roche), or with napthoic acid derivatives. Naphthoic acid derivatives, such as, in particular, 6-[3-(1-adamantyl)-4-methoxyphenyl]-2-naphthoic acid, commonly known as adapalene (the product Differin® marketed by Galderma), are widely described and recognized as active principles which are as effective as tretinoin in the treatment of acne. In addition, adapalene exhibits the advantage of causing fewer side effects, such as phenomena of irritation, of drying of the skin or of intolerance, than the other active principles described above, which makes it a product of choice.

Various therapeutic combinations, such as various associations of therapeutic treatments, have been envisaged for the treatment of dermatological disorders and in particular, acne. However, it may transpire that certain combinations combine the undesirable effects of each of the therapeutic active principles or exacerbate one of the harmful side effects of one of the active principles administered; in yet other cases, it may transpire that one of the active principles no longer exhibits its optimum therapeutic activity in the presence of another active principle; which makes these associated uses or uses in combination non-advantageous, indeed even inappropriate.

Attempts have also been made to develop compositions which make it possible to adjust the topical penetration of certain active principles by including, in compositions, compounds of the type consisting of polyurethane polymers or their derivatives (EP 0 299 758). The product Avita®, marketed by Bertek Pharmaceuticals Inc., is an example thereof. It comprises, in particular, 0.025% by weight, with respect to the total weight of the composition, of tretinoin dissolved in compositions of gel or cream type which comprise polyurethane polymers (polyol pre-polymers of type 2, marketed by Bertek Pharmaceuticals Inc.) for the purpose of limiting desquamation, irritation and desiccation of the skin.

Thus, adapalene (category of the retinoids) is commonly used in the treatment of moderate acne, as well as benzoyl peroxide. Their respective effectiveness is universally recognized. The combined application of adapalene and benzoyl peroxide might prove to be a treatment of choice in making possible the treatment of moderate to severe acne. However, it is necessary to anticipate an irritant effect of this composition on the skin due to the joint presence of the two molecules, which are known to be irritants.

### SUMMARY OF THE INVENTION

Thus, the present invention features particularly effective compositions for topical application, comprising benzoyl peroxide and a naphthoic acid compound, without eliciting an obviously irritant effect which prevents same from being used in the relatively long term by the subject.

Thus, it has now surprisingly been demonstrated, that benzoyl peroxide, employed in a form encapsulated in a polymeric system of porous particles, in combination with at least one naphthoic acid compound, such as adapalene, makes it possible to increase the penetration of these two active principles and also makes it possible to reduce cutaneous irritation, in comparison with the same combination comprising the said naphthoic acid compound and benzoyl peroxide in the free form.

### BRIEF DESCRIPTION OF THE DRAWING

The FIGURE of Drawing shows the results of a study of tolerance in the BALB/c mouse and comparing the irritant power of several reference gels.

### DETAILED DESCRIPTION OF BEST MODE AND SPECIFIC/PREFERRED EMBODIMENTS OF THE INVENTION

Specifically, an indirect effect of the encapsulation of benzoyl peroxide has now been demonstrated on the activity of the naphthoic acid compound: in particular, the cutaneous penetration of the said derivative and the irritation caused by the latter are reduced by the encapsulated benzoyl peroxide.

In point of fact, nothing in the prior art would suggest that benzoyl peroxide, used in encapsulated form, could increase the cutaneous penetration not only of encapsulated benzoyl peroxide but also of the other active principle present in the said combination. Nor did the prior art suggest better tolerance of the compositions according to the invention in comparison with an equivalent composition where the benzoyl peroxide is in the free form and thus non-encapsulated. Specifically, the publication by R. C. Wester et al. (“Controlled release of benzoyl peroxide from a porous microsphere polymeric system can reduce topical irritancy”, R. C. Wester et al., JAAD, vol. 24, 5, May 1991, pp. 720-726), suggests that the reduction in the irritation of encapsulated benzoyl peroxide is essentially due to the controlled release of the active principle and thus to a reduction in the rate of cutaneous penetration of the active principle.

In view of the above, the present invention features administration of benzoyl peroxide in the form encapsulated in a polymeric system of porous particles as an agent for increasing the cutaneous penetration of the said benzoyl peroxide and/or of at least one naphthoic acid derivative/compound,

and/or for reducing the cutaneous irritation of the said benzoyl peroxide and/or of at least one naphthoic acid compound, in particular, for formulating a medicament useful to reduce the cutaneous irritation caused by at least one naphthoic acid derivative.

The present invention features compositions comprising benzoyl peroxide and at least one naphthoic acid compound in high amounts. The compositions according to the invention result in good penetration of the active principles while retaining tolerance thereto. In point of fact, it is, surprisingly, by employing benzoyl peroxide in a form encapsulated in a polymeric system of porous particles, together with the naphthoic acid, compositions according to the invention are provided exhibiting the desired advantages when they are applied topically to the skin. The compositions according to the invention prove in particular, to be particularly effective while exhibiting very good tolerance.

Thus, this invention features the use of a polymeric system of porous particles as an agent for increasing the cutaneous penetration of benzoyl peroxide and/or of at least one naphthoic acid compound,

and/or for reducing the cutaneous irritation of the said benzoyl peroxide and/or of at least one naphthoic acid compound. According to the invention, the benzoyl peroxide is in a form encapsulated in the polymeric system of porous particles.

This is because, according to the invention, the benzoyl peroxide in the form encapsulated in a polymeric system of porous particles makes it possible to increase the cutaneous penetration of the benzoyl peroxide but also of the naphthoic acid derivative, as is shown in Example 4 below. Furthermore, the compositions comprising encapsulated benzoyl peroxide, and also a naphthoic acid derivative, are well tolerated in the mouse or in man, as is shown in Examples 5 and 6 to follow.

The present invention also features compositions for topical application comprising, in a physiologically acceptable medium, benzoyl peroxide in the form encapsulated in a polymeric system of porous particles and at least one naphthoic acid compound, the said naphthoic acid compound being present in an amount greater than or equal to 0.2% by weight, with respect to the total weight of the said composition.

The term “physiologically acceptable vehicle” means a vehicle compatible with the skin, mucous membranes and/or superficial body growths.

Such a composition can be used as medicament, in particular, in the preparation of a pharmaceutical composition intended for the treatment and/or prevention of dermatological conditions related to a disorder of keratinization relating to cell differentiation and to cell proliferation, in particular, in treating comedonal acne, acne vulgaris, papulocomedonal acne, nodulocystic acne, polymorphous acne, acne rosacea, acne conglobata, senile acne or secondary acne, such as solar acne, acne medicamentosa or occupational acne.

The compositions according to the invention thus comprise at least one naphthoic acid compound of retinoid type.

Naphthoic acid is a compound having the following formula:

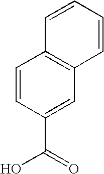

The term “naphthoic acid derivative” or “compound” means the compounds of formula (I):

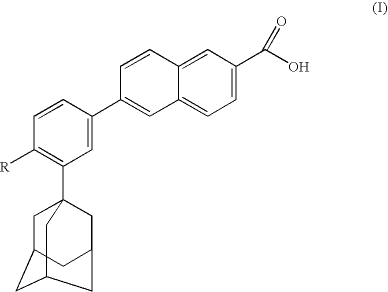

wherein:

R is a hydrogen atom, a hydroxyl radical, a linear or branched alkyl radical having from 1 to 4 carbon atoms, an alkoxy radical having from 1 to 10 carbon atoms or a cycloaliphatic radical which is unsubstituted or substituted.

The term “linear or branched alkyl radical having 1 to 4 carbon atoms” means preferably the methyl, ethyl, propyl and butyl radicals.

The term “alkoxy radical having from 1 to 10 carbon atoms” means preferably the methoxy, ethoxy, propoxy, butoxy, hexyloxy and decyloxy radicals.

The term “cycloaliphatic radical” means preferably mono- or polycyclic radicals, such as the 1-methylcyclohexyl radical or the 1-adamantyl radical.

The selection will advantageously be made, among the naphthoic acid compounds suitable for the compositions according to the invention, of 6-[3-(1-adamantyl)-4-methoxyphenyl]-2-naphthoic acid (adapalene), 6-[3-(1-adamantyl)-4-hydroxyphenyl]-2-naphthoic acid, 6-[3-(1-adamantyl)-4-decyloxyphenyl]-2-naphthoic acid and 6-[3-(1-adamantyl)-4-hexyloxyphenyl]-2-naphthoic acid. Preferably, the naphthoic acid derivative/compound is 6-[3-(1-adamantyl)-4-methoxyphenyl]-2-naphthoic acid or adapalene.

The abovementioned naphthoic acid derivatives are generally provided in a form dispersed in the composition according to the invention. The insoluble naphthoic acid derivatives are thus distributed homogeneously in the composition according to the invention.

According to the invention, the naphthoic acid compounds are employed at concentrations generally of less than or equal to 10% by weight, with respect to the total weight of the composition, and are thus preferably from 0.2% to 10% by weight, with respect to the total weight of the composition, and preferably from 0.25% to 5% by weight, or preferably from 0.3% to 3% by weight, and very preferably is 0.3% by weight, with respect to the total weight of the composition.

Throughout the present text, unless otherwise specified, it is understood that, when ranges of concentrations are given, they include the upper and lower limits of the said range.

Advantageously, the naphthoic acid derivative included in the compositions according to the invention is adapalene. The concentration of the adapalene in the compositions according to the invention is preferably from 0.2% to 0.5%, more preferably from 0.25% to 0.35%, in particular, at a concentration of 0.3%.

The compositions according to the invention also comprise benzoyl peroxide in the form encapsulated in a polymeric system comprised of porous particles.

Benzoyl peroxide in the form encapsulated in a polymeric system of porous particles is described, in particular, in U.S. Pat. No. 5,879,716. The porous particles are generally solid and comprise pores open towards the outside and an impregnating agent, in this instance benzoyl peroxide, retained inside the said pores. The impregnating agent is optionally present with a solvent.

The solid particles are generally spherical in shape. They are advantageously formed by suspension polymerization in the presence of a pore-forming agent, followed by removal of the pore-forming agent from the pores thus formed and impregnation with the said impregnating agent. The mean diameter of the particles is generally from approximately 10 to approximately 40 μm, with advantageously a total volume of the pores from approximately 0.01 (preferably 0.1) ml/g to approximately 4.0 (preferably 2) ml/g, having a surface area from approximately 1 (preferably 20) m²/g to approximately 500 (preferably 200) m²/g and having a mean diameter of the pores from approximately 0.001 (preferably 0.003) to approximately 3.0 (preferably 1.0) μm. The polymerization is thus advantageously carried out in suspension in a liquid/liquid system, such as described, in particular, in U.S. Pat. No. 5,879,716. The polymers thus formed are copolymers of styrene and of divinylbenzene; or of methyl methacrylate and of ethylene glycol dimethacrylate; or of 4-vinylpyridine and of ethylene glycol dimethacrylate. Copolymers of methyl methacrylate and of ethylene glycol dimethacrylate are particularly preferred. Exemplary is the product marketed by the group Cardinal Health under the trademark of Microsponge® P009A Benzoyl Peroxide.

The content of benzoyl peroxide in the polymeric system is generally, by dry weight, from 35 and 55%, preferably from 38 and 51%, with respect to the total weight of the polymeric system impregnated with benzoyl peroxide. The content of volatile compounds (such as water) optionally present in the impregnated polymeric system is from 0 and 10% by weight, more specifically from 5 and 10% by weight, with respect to the total weight of the said impregnated system.

In the compositions according to the invention, the encapsulated benzoyl peroxide is included at concentrations generally of less than or equal to 20% by weight, with respect to the total weight of the composition, and preferably at concentrations of from 1.0% to 10% by weight, with respect to the total weight of the composition, and preferentially from 1.5% to 6% by weight, more preferably from 2.0% to 5% by weight.

The amounts specified above thus allow benzoyl peroxide in the encapsulated form to increase the cutaneous penetration of the said combination, in particular, of the said naphthoic acid compound and/or of the benzoyl peroxide, and/or to reduce the irritation of the said combination, in particular, of the said naphthoic acid compound.

Particularly preferably, the ratio of the amount of naphthoic acid to the encapsulated benzoyl peroxide according to the invention is from 0.01 to 0.5, preferably from 0.01 to 0.2 and particularly preferably from 0.02 to 0.15.

The compositions of the present invention can be provided in all the formulation forms normally employed for topical application, in particular, in the form of aqueous, aqueous/alcoholic or oily dispersions, of dispersions of the lotion type, of aqueous, anhydrous or lipophilic gels, of emulsions with a liquid or semi-liquid consistency of the milk type, obtained by dispersion of a fatty phase in an aqueous phase (O/W) or vice versa (W/O), or of suspensions or emulsions with a soft, semi-liquid or solid consistency of the cream, cream gel or ointment type, or also of microemulsions, of microcapsules, of microparticles or of vesicular dispersions of ionic and/or nonionic type, or also in the foam form.

Preferably, the compositions according to the invention are provided in the form of lotions, cream gels, gels, creams or foams.

One skilled in the art will take care to select the excipients constituting the compositions according to the invention as a function of the formulation form desired and such that the advantageous properties of the composition according to the invention are retained.

In addition, the compositions according to the invention can in particular, comprise one or more of the following ingredients:

a) one or more gelling agents or suspending agents,

b) one or more chelating agents,

c) one or more wetting agents,

d) one or more preservatives,

e) one or more emulsifiers.

Exemplary are, of gelling agents or suspending agents which can be included in the compositions according to the invention, of carbomers marketed under the generic name of Carbopol®, carbomers said to be insensitive to electrolytes, marketed under the trademark of Ultrez 10® or of Carbopol ETD® by BF Goodrich, polysaccharides, with, as non-limiting examples, xanthan gum, such as Keltrol T®, marketed by Kelco, guar gum, chitosans, cellulose and its derivatives, such as hydroxyethylcellulose, in particular, the product marketed under the trademark of Natrosol HHX 250® by Aqualon, and the copolymer of acrylamide and of sodium acrylamido-2-methylpropanesulfonate as a 40% dispersion in isohexadecane and polysorbate 80, marketed under the trademark of Simulgel 600® by Seppic, or in isoparaffin and polysorbate 80, marketed under the trademark of Sepigel 305 by Seppic.

Exemplary are, as preferred gelling agent, of carbomers, marketed in particular, under the trademarks Carbopol 974P NF and Carbopol 980 NF.

Exemplary are, among chelating agents, as non-limiting examples, ethylenediaminetetraacetic acid (EDTA), diethylenetriaminepentaacetic acid (DTPA), ethylenediaminedi(o-hydroxyphenylacetic acid) (EDDHA), (2-hydroxyethyl)ethylenediaminetriacetic acid (HEDTA), ethylenediaminedi(o-hydroxy-p-methylphenylacetic acid) (EDDHMA) and ethylenediaminedi(5-carboxy-2-hydroxyphenylacetic acid) (EDDCHA).

Exemplary, as preferred chelating agent, is ethylenediaminetetraacetic acid (EDTA), marketed in particular, under the trademark Titriplex III®.

Preferably, wetting agents are employed, the role of which is to reduce the surface tension from the dispersed particles and the dispersing medium and to thus make possible greater spreading of the liquid. Exemplary such compounds are propylene glycol, dipropylene glycol, propylene glycol dipelargonate, lauroglycol and ethoxydiglycol, alone or as a mixture.

Exemplary, as preferred wetting agent, is propylene glycol. Also exemplary of such wetting agents are compounds of the family of the Poloxamers and more particularly Poloxamer 124 and/or Poloxamer 182.

Exemplary preservatives are benzoic acid and its derivatives with benzyl alcohol, benzalkonium chloride, sodium benzoate, bronopol, chlorhexidine, chlorocresol and its derivatives, ethyl alcohol, phenethyl alcohol, phenoxyethanol, potassium sorbate, diazolidinylurea, parabens, such as propylparaben or methylparaben, taken alone or as mixtures.

Exemplary preferred preservatives are parabens and phenoxyethanol or benzalkonium chloride, alone or as mixtures.

The compositions according to the invention can comprise one or more emulsifiers.

The surfactants can be classified, according to their structure, under the generic terms “ionic” (anionic, cationic or amphoteric) or “nonionic”. Nonionic surfactants are surfactants which do not dissociate into ions in water and are thus insensitive to variations in pH.

Nonionic surfactants are particularly well suited for the preparation of emulsions of oil-in-water type, which comprise compositions according to the present invention. Exemplary nonionic surfactants are those exhibiting a high HLB, such as sorbitan esters, such as POE(20) sorbitan monooleate, marketed under the trademark of “Tween 80” (HLB=15); POE(20) sorbitan monostearate, marketed under the trademark of “Tween 60” (HLB=14.9); ethers of fatty alcohols, such as POE(21) stearyl ether (HLB=15.5); or ceteareth-20, marketed under the trademark of “Eumulgin B2” by Cognis (HLB of 15.5). Examples of nonionic surfactants with a low HLB (lipophilic surfactants) are sorbitan esters, such as sorbitan monostearate (marketed under the trademark of Span 60 by Uniqema), glycerol esters (marketed under the trademark of Cutina GMSVPH by Cognis), such as glycerol monostearate (Cutina GMS from Cognis), or sucrose esters with a low HLB, such as sucrose distearate. The nonionic surfactants can be used alone or as a mixture of two or more of same in order to form the emulsifying system comprising the emulsions of the invention.

Preferred emulsifiers are hydrophilic emulsifiers of the type Tween 80, Glyceryl Monostearate & POE Stearate, marketed under the trademark Arlacel 165FL® by Uniqema; or lipophilic emulsifiers of the type Glucate SS and Glucamate SSE, Polyoxyethylene (21) Stearyl Ether, marketed under the trademark Brij 721® by Uniqema.

The compositions according to the invention can also comprise a fatty phase. This fatty phase can comprise, for example, vegetable, mineral, animal or synthetic oils, silicone oils and their mixtures.

Exemplary are, as examples of mineral oil, liquid paraffins of different viscosities, such as Primol 352®, Marcol 82® or Marcol 152®, marketed by Esso.

Exemplary vegetable oils are sweet almond oil, palm oil, soybean oil, sesame oil or sunflower oil.

Exemplary animal oils are lanolin, squalene, fish oil or mink oil with, as derivative, the squalane marketed under the trademark Cosbiol® by Laserson.

Exemplary synthetic oils are esters, such as cetearyl isononanoate, for example, the product marketed under the trademark of Cetiol SN® by Cognis France, diisopropyl adipate, for example, the product marketed under the trademark of Ceraphyl 230® by ISF, isopropyl palmitate, for example, the product marketed under the trademark of Crodamol IPP® by Croda, or caprylic/capric triglyceride, such as Miglyol 812®, marketed by Hüls/Lambert Rivière.

Exemplary silicone oils are dimethicones, such as the product marketed under the trademark of Dow Corning 200 Fluid®, or a cyclomethicone, such as the product marketed under the trademark of Dow Corning 244 Fluid® by Dow Corning or the product marketed under the trademark of Mirasil CM5® by SACI-CFPA.

Solid fatty substances are also exemplary, such as natural or synthetic waxes. In this case, one skilled in the art will adjust the heating temperature of the preparation according to whether or not these solids are present.

For the compositions according to the invention, liquid paraffins, more particularly Marcol 152®, and synthetic oils, more particularly Miglyol 812®, are preferred.

The compositions of the invention can additionally comprise any additive normally used in the cosmetics or pharmaceutical field, such as neutralizing agents, sunscreens, antioxidants, fillers, electrolytes, colorants, normal inorganic or organic bases or acids, fragrances, essential oils, cosmetic active principles, moisturizing agents, vitamins, essential fatty acids, sphingolipids, self-tanning compounds, such as DHA, soothing and skin-protecting agents, such as allantoin, propenetrating agents, or a mixture of these. Of course, one skilled in the art will take care to select this or these optional additional compounds and/or their amounts such that the advantageous properties of the compositions according to the invention are not, or not substantially, detrimentally affected.

These additives can be present in the composition in a proportion of 0.001% to 20% by weight, with respect to the total weight of the composition.

In one specific embodiment of the invention, the composition is provided in the form of an aqueous gel.

In a specific embodiment of the invention, the composition comprises:

0.1 to 90% of water;

0.01 to 10% of a gelling agent or of a combination of gelling agents;

0.01 to 20% of propenetrating agent;

0.01 to 1% of chelating agent;

0.01 to 10% of wetting agent;

0.2 to 1% of at least one naphthoic acid derivative/compound;

0.001 to 30% of benzoyl peroxide encapsulated in microspheres.

The present invention also features administration of the compositions as described above as medicaments, whether regime or regimen.

The present invention also features administration of the novel compositions as described above in cosmetics and in dermatology.

In particular, this invention features pharmaceutical compositions useful for the treatment and/or prevention of dermatological conditions/afflictions related to a disorder of keratinization relating to cell differentiation and to cell proliferation, in particular, in treating acne vulgaris, comedonal acne, papulopustular acne, papulocomedonal acne, nodulocystic acne, acne conglobata, acne keloid of the back of the neck, recurrent acne miliaria, acne necrotica, acne neonatorum, occupational acne, acne rosacea, senile acne, solar acne and acne medicamentosa.

More particularly, the present invention features pharmaceutical compositions useful to prevent and/or treat acne vulgaris.

Preferably, the said compositions according to the invention are administered topically.

In addition, the present invention also features the cosmetic application of a composition according to the invention in the treatment of skin having a tendency towards acne, in order to combat the greasy appearance of the skin or hair, in protecting same from the harmful effects of the sun or in the treatment of physiologically dry skin, or to prevent and/or combat photo-induced or chronological aging.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Formulation of Gel Type

The following formulation is prepared.

| Constituents | Content (% w/w) |
|---|---|
| Adapalene | 0.10 |
| Poloxamer 124 | 0.20 |
| Propylene glycol | 4.00 |
| Encapsulated benzoyl peroxide* | 6.15 |
| (peroxide content 2.5%) | |
| Glycerol | 4.00 |
| Carbomer | 0.85 |
| Silica | 0.02 |
| Sodium docusate | 0.05 |
| Disodium EDTA | 0.10 |
| Sodium hydroxide | q.s. for pH 5.5 ± 0.5 |
| Purified water | q.s. for 100 |

*Microsponge ® P009A Benzoyl Peroxide from APS

Procedure:

The water, the EDTA, the sodium docusate, the silica, the carbomer and the glycerol are introduced into the formulary beaker and the mixture is stirred with a Rayneri mixer equipped with a deflocculating paddle until dissolution is complete (speed 500-700 rev/min).

The BPO microsponges are subsequently added.

The propylene glycol, the Poloxamer and the adapalene are introduced into a supplementary beaker and the mixture is stirred using an Ultra-Turrax homogenizer (20,000 rev/min).

The active phase is introduced into the formulary beaker with stirring.

The formulation is then neutralized.

Specifications at T0:

Macroscopic appearance: White, smooth and glossy product
Microscopic appearance: Good dispersion of the two active principles.

### Example 2

### Formulation of Gel Type

The following formulation is prepared:

| Constituents | Content (% w/w) |
|---|---|
| Adapalene | 0.10 |
| Poloxamer 124 | 0.20 |
| Propylene glycol | 4.00 |
| Encapsulated benzoyl peroxide* | 6.15 |
| (i.e., a peroxide content of 2.5%) | |
| Xanthan gum | 0.50 |
| Hydroxyethyl cellulose | 1.50 |
| Silica | 0.021 |
| Magnesium aluminum silicate | 2.00 |
| Glycerol-comprising microsponges | 4.00 |
| Glycerol | 2.00 |
| Sodium docusate | 0.05 |
| Disodium EDTA | 0.10 |
| Purified water | q.s. for 100 |

*Microsponge ® P009A Benzoyl Peroxide from APS

Procedure:

The water, the glycerol and the EDTA are introduced into the formulary beaker and the mixture is stirred with a Rayneri mixer equipped with a deflocculating paddle while heating at 60° C. until the mixture has dissolved (speed 500-700 rev/min).

The magnesium aluminum silicate is subsequently added, followed by the xanthan gum and the hydroxyethyl cellulose and then the benzoyl peroxide microsponges, and the mixture is maintained under stirring until completely dispersed.

The sodium docusate, dissolved beforehand in the propylene glycol, is added at 40° C.

The propylene glycol, the Poloxamer and the adapalene are introduced into a supplementary beaker and the mixture is stirred with an Ultra-Turrax homogenizer (20,000 rev/min).

This active phase is introduced into the formulary beaker with stirring.

Specifications at T0:

Macroscopic appearance: Off-white to beige, smooth and glossy product
Microscopic appearance: Good dispersion of the two active principles.

### Example 3

### Formulation of Gel Type

The following formulation is prepared:

| Constituents | Content (% w/w) |
|---|---|
| Adapalene | 0.30 |
| Poloxamer 124 | 0.20 |
| Propylene glycol | 4.00 |
| Encapsulated benzoyl peroxide* | 6.15 |
| (i.e., a peroxide content of 2.5%) | |
| Xanthan gum | 0.50 |
| Hydroxyethyl cellulose | 1.50 |
| Silica | 0.021 |
| Magnesium aluminum silicate | 2.00 |
| Glycerol-comprising microsponges | 4.00 |
| Glycerol | 2.00 |
| Sodium docusate | 0.05 |
| Disodium EDTA | 0.10 |
| Purified water | q.s. for 100 |

*Microsponge ® P009A Benzoyl Peroxide from APS

Procedure:

The water, the glycerol and the EDTA are introduced into the formulary beaker and the mixture is stirred with a Rayneri mixer equipped with a deflocculating paddle while heating at 60° C. until the mixture has dissolved (speed 500-700 rev/min).

The magnesium aluminum silicate is subsequently added, followed by the xanthan gum and the hydroxyethyl cellulose and then the benzoyl peroxide microsponges, and the mixture is maintained under stirring until completely dispersed.

The sodium docusate, dissolved beforehand in the propylene glycol, is added at 40° C.

The propylene glycol, the Poloxamer and the adapalene are introduced into a supplementary beaker and the mixture is stirred with an Ultra-Turrax homogenizer (20,000 rev/min).

This active phase is introduced into the formulary beaker with stirring.

Specifications at T0:

Macroscopic appearance: Off-white to beige, smooth and glossy product
Microscopic appearance: Good dispersion of the two active principles.

### Example 4

### Study of In Vitro Release/Penetration

The goal of the present study is to compare in vitro the release/penetration through the human skin without occlusion of the active principles formulated at 0.1% of adapalene (w/w) and at 2.5% (w/w) of benzoyl peroxide in the encapsulated form in a gel similar to that described in Example 1 (formulation A). Comparative studies are carried out with similar formulations, but in which:

the encapsulated benzoyl peroxide is replaced with benzoyl peroxide in the free form (formulation B),

the encapsulated benzoyl peroxide and the adapalene are replaced with adapalene alone at 0.1% (w/w) (formulation C),

the encapsulated benzoyl peroxide and the adapalene are replaced with benzoyl peroxide alone and in the free form at 2.5% (w/w) (formulation D).

The absorption studies were carried out using excised human skin mounted under static conditions for a period of 16 hours. Three samples of skin taken from women (aged 68 years) were used. An amount of 10 mg of each formulation was applied to a skin surface area of 1 cm². The concentrations of adapalene and benzoyl peroxide in the fluid fractions collected over time and remaining in the skin at the end of the study were evaluated by the HPLC method with fluorescence detection (based on a validated method. Quantification limit: 1 ng·ml⁻¹).

The experimental results are reported in the table below:

| | Formulation | Formulation | Formulation | Formulation |
|---|---|---|---|---|
| | A | B | C | D |
| Total | 1.2 μg/cm² | 0.5 μg/cm² | 0.4 μg/cm² | NA |
| penetrated | | | | |
| amount of | | | | |
| adapalene | | | | |
| Total | 35.8 μg/cm² | 12.4 μg/cm² | NA | 13.6 μg/cm² |
| penetrated | | | | |
| amount of | | | | |
| benzoyl | | | | |
| peroxide | | | | |

It should be noted that, whatever the formulation tested, the adapalene is distributed mainly in the epidermis (including stratum corneum), while the benzoyl peroxide is encountered throughout all the layers of the skin and in the reservoir. The total penetrated amount of adapalene is from 2 to 3 times higher in formulation A than in formulations B and C. The total penetrated amount of benzoyl peroxide is approximately 3 times higher in formulation A than in formulations B and D. These results thus demonstrate that a combination of adapalene and of benzoyl peroxide in the encapsulated form makes it possible to increase the percutaneous penetration of the two pharmaceutical active principles.

### Example 5

### Study of Tolerance in the BALB/c Mouse

The goal of the present study is to compare the irritant power of a reference gel comprising 0.1% of adapalene alone or comprising benzoyl peroxide (BPO) alone with that of several formulations of 0.1% adapalene in combination with free or encapsulated benzoyl peroxide on the skin of the ear of the BALB/c mouse after topical applications repeated for 12 days.

The daily topical application (20 μl) of the test products is carried out on the internal face of the ear of BALB/c mice divided into ten groups (female mice approximately 8 weeks old) at the rate of one application per day for 12 days. The test products are:

Group 1: vehicle (control)

Group 2: 0.1% adapalene

Group 3: 2.5% free BPO

Group 4: 5% free BPO

Group 5: 0.1% adapalene+2.5% free BPO

Group 6: 0.1% adapalene+5% free BPO

Group 7: 2.5% BPO encapsulated in microsponges

Group 8: 5% BPO encapsulated in microsponges

Group 9: 0.1% adapalene+2.5% BPO encapsulated in microsponges

Group 10: 0.1% adapalene+5% BPO encapsulated in microsponges

Evaluation is carried out by measurements of the thickness of the ear by means of the Oditest and by clinical observation of the animals from the 1^{st} to 21^{st} day.

The results are represented in the FIGURE of Drawing.

The topical application of the vehicle does not cause irritation on the mouse ear.

These results show that the application of 5% free BPO (group 4) causes an increase of 31% in the AUC, thus in the thickness of the ear and in the irritation, with respect to the vehicle alone (group 1), while the application of 2.5% or 5% BPO in the encapsulated form (groups 7 and 8) does not cause irritation.

Adapalene alone (group 2) causes significant irritation and the AUC is increased by 59% with respect to the vehicle alone.

When this active principle is combined with 2.5% or 5% free BPO (groups 5 and 6), the increase in the AUC is 14%, with respect to adapalene alone (group 2), and thus the irritation is significant.

On the other hand, when adapalene is combined with 2.5% or 5% BPO in the encapsulated form (groups 9 and 10), the AUC is reduced, not significantly, by 6%, with respect to adapalene alone (group 2).

Conclusions:

Adapalene alone, formulated in the vehicle, causes irritation after 2 weeks of topical treatment;

BPO in the free form at 5% causes irritation after 9 applications;

the 0.1% adapalene+free BPO combination causes greater irritation than that of adapalene alone;

the 0.1% adapalene+encapsulated BPO combination causes slightly lower irritation than that of adapalene alone.

### Example 6

### Study of Tolerance in Man

The goal of the present study is to evaluate the cutaneous tolerance of compositions comprising adapalene and BPO in the encapsulated form in man, in comparison with compositions comprising adapalene and BPO in the free form.

This study was carried out under double blind conditions and with intra-individual comparison (left/right of the face) on 30 healthy subjects for 21 days. The subjects were randomized into 3 groups of 10 subjects according to the following table; the applications (left/right) were randomized for each group:

| | Number | | |
|---|---|---|---|
| | of | Product applied to a | Product applied to the |
| Group | subjects | half-face | other half-face |
| 1 | 10 | 0.1% adapalene + 2.5% | 0.1% adapalene + 5% |
| | | encapsulated BPO | encapsulated BPO |
| 2 | 10 | 0.1% adapalene + 2.5% | 0.1% adapalene + 2.5% |
| | | encapsulated BPO | free BPO |
| 3 | 10 | 0.1% adapalene + 5% | 0.1% adapalene + 2.5% |
| | | encapsulated BPO | free BPO |

The cutaneous reactions were evaluated on each side of the face before each application. Cutaneous erythema, desquamation and dryness were evaluated by the investigator. Sensations of burning and pruritus were evaluated by the subject using a scale of 4 values (0=none; 3=severe). In the case of severe irritation on one of the sides of the face (assessed by the investigator in the light of the clinical signs and/or of the symptoms described by the subject), the application was interrupted on both sides of the face.

The first criterion is the safety score (SSS=Safety Sum Score) of the following signs/symptoms: erythema, desquamation, dryness, burning and pruritus.

This score is subjected to an analysis of variance. The following results were obtained:

| | |
|---|---|
| Group | SSS +/− sem |

| | | |
|---|---|---|
| 1 | 0.1% adapalene + 2.5% | 2.23 ± 0.49 |
| | encapsulated BPO | |
| | 0.1% adapalene + 5% | 2.41 ± 0.48 |
| | encapsulated BPO | |
| 2 | 0.1% adapalene + 2.5% | 1.86 ± 0.34 |
| | encapsulated BPO | |
| | 0.1% adapalene + 2.5% free BPO | 2.48 ± 0.37 |
| 3 | 0.1% adapalene + 5% | 2.66 ± 0.33 |
| | encapsulated BPO | |
| | 0.1% adapalene + 2.5% free BPO | 2.77 ± 0.26 |

In terms of cutaneous tolerance, the analysis of the SSS shows that:

the adapalene+2.5% encapsulated BPO combination causes significantly less irritation than the adapalene+2.5% free BPO combination;

the adapalene+5% encapsulated BPO combination does not differ significantly from the adapalene+2.5% free or encapsulated BPO combinations.

No significant side effect was reported during the study. The adapalene+2.5% free or 5% encapsulated BPO combinations were associated with more cutaneous side effects than the adapalene+2.5% encapsulated BPO combination.

Thus, the formulation comprising adapalene and 2.5% BPO encapsulated in microsponges improves the BPO tolerance, as is shown by the SSS and the less frequent side effects.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable, reduced-irritant dermatological/cosmetic composition comprising at least one naphthoic acid compound and an amount of benzoyl peroxide encapsulated in a polymeric system comprised of porous particles for increasing cutaneous penetration of said at least one naphthoic acid compound, formulated into a topically applicable, physiologically acceptable medium therefor.

2. The dermatological/cosmetic composition as defined by claim 1, said at least one naphthoic acid compound having the formula (I): 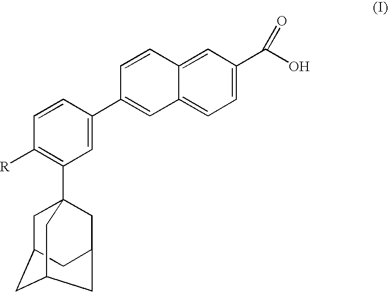
wherein R is a hydrogen atom, a hydroxyl radical, a linear or branched alkyl radical having from 1 to 4 carbon atoms, an alkoxy radical having from 1 to 10 carbon atoms or a cycloaliphatic radical.

3. The dermatological/cosmetic composition as defined by claim 2, said at least one naphthoic acid compound being selected from the group consisting of 6-[3-(1-adamantyl)-4-methoxyphenyl]-2-naphthoic acid, 6-[3-(1-adamantyl)-4-hydroxyphenyl]-2-naphthoic acid, 6-[3-(1-adamantyl)-4-decyloxyphenyl]-2-naphthoic acid and 6-[3-(1-adamantyl)-4-hexyloxyphenyl]-2-naphthoic acid.

4. The dermatological/cosmetic composition as defined by claim 3, said at least one naphthoic acid compound comprising 6-[3-(1-adamantyl)-4-methoxyphenyl]-2-naphthoic acid.

5. The dermatological/cosmetic composition as defined by claim 1, wherein the porous particles of said polymeric system are selected from the group consisting of copolymers of styrene and of divinylbenzene;
copolymers of methyl methacrylate and of ethylene glycol dimethacrylate;
and copolymers of 4-vinylpyridine and of ethylene glycol dimethacrylate.

6. The dermatological/cosmetic composition as defined by claim 1, wherein the content of benzoyl peroxide in the polymeric system ranges, by dry weight, from 35% to 55%, with respect to the total weight of the polymeric system impregnated with benzoyl peroxide.

7. The dermatological/cosmetic composition as defined by claim 1, wherein the ratio of the amount of naphthoic acid compound to the encapsulated benzoyl peroxide ranges from 0.01 to 0.5.

8. The dermatological/cosmetic composition as defined by claim 1, wherein the amount of said at least one naphthoic acid compound therein is greater than or equal to 0.2% by weight, with respect to the total weight thereof.

9. The dermatological/cosmetic composition as defined by claim 8, wherein the concentration of said at least one naphthoic acid compound ranges from 0.2% to 10% by weight, with respect to the total weight thereof.

10. The dermatological/cosmetic composition as defined by claim 4, wherein the concentration of said 6-[3-(1-adamantyl)-4-methoxyphenyl]-2-naphthoic acid is approximately 0.3% by weight, with respect to the total weight thereof.

11. The dermatological/cosmetic composition as defined by claim 1, wherein the amount of benzoyl peroxide ranges from 1.0% to 10% by weight, with respect to the total weight thereof.

12. A regime or regimen for the treatment of a keratinization disorder, comprising topically applying onto the affected skin area of an individual in need of such treatment, a thus effective amount of the dermatological/cosmetic composition as defined by claim 1.

13. A regime or regimen for treating acne vulgaris, comedonal acne, papulopustular acne, papulocomedonal acne, nodulocystic acne, acne conglobata, acne keloid of the back of the neck, recurrent acne miliaria, acne necrotica, acne neonatorum, occupational acne, acne rosacea, senile acne, solar acne or acne medicamentosa, comprising topically applying onto the affected skin area of an individual in need of such treatment, a thus effective amount of the dermatological/cosmetic composition as defined by claim 1.

14. A regime or regimen for the prevention or treatment of acne vulgaris, comprising topically applying onto the affected skin area of an individual in need of such treatment, a thus effective amount of the dermatological/cosmetic composition as defined by claim 1.

15. A regime or regimen for the treatment of skin having a tendency towards acne, in order to combat the greasy appearance of the skin or hair, or for protecting the skin from the harmful effects of the sun or in the treatment of physiologically dry skin, or to prevent and/or combat photo-induced or chronological aging of the skin, comprising topically applying onto the affected skin area of an individual in need of such treatment, a thus effective amount of the dermatological/cosmetic composition as defined by claim 1.

16. The dermatological/cosmetic composition as defined by claim 1, further comprising:
a) one or more gelling agents or suspending agents,
b) one or more chelating agents,
c) one or more wetting agents,
d) one or more preservatives,
e) one or more emulsifiers.

17. The dermatological/cosmetic composition as defined by claim 1, formulated as a lotion, cream gel, gel, cream or foam.

18. The dermatological/cosmetic composition as defined by claim 1, further comprising at least one additive selected from the group consisting of neutralizing agents, sunscreens, antioxidants, fillers, electrolytes, colorants, normal inorganic or organic bases or acids, fragrances, essential oils, cosmetic active principles, moisturizing agents, vitamins, essential fatty acids, sphingolipids, self-tanning compounds, soothing and skin-protecting agents, propenetrating agents, and mixture thereof.

19. The dermatological/cosmetic composition as defined by claim 1, comprising:
0.1 to 90% of water;
0.01 to 10% of a gelling agent or combination of gelling agents;
0.01 to 20% of propenetrating agent;
0.01 to 1% of chelating agent;
0.01 to 10% of wetting agent;
0.2 to 1% of at least one naphthoic acid compound;
0.001 to 30% of benzoyl peroxide encapsulated in microspheres.

20. The dermatological/cosmetic composition as defined by claim 1, formulated as an aqueous gel.
